(19) United States
(12) Patent Application Publication
(10) Pub. No.: US 2022/0143016 A1
(43) Pub. Date: May 12, 2022

(54) METHOD FOR TREATING A MULTIPLE MYELOMA

(71) Applicant: PTC THERAPEUTICS, INC., South Plainfield, NJ (US)

(72) Inventors: Marla L. Weetall, Morristown, NJ (US); Liangxian Cao, East Brunswick, NJ (US); Arnold Bolomsky, Scheiblwies (AT); Heinz Ludwig, Vienna (AT)

(21) Appl. No.: 17/433,106

(22) PCT Filed: Feb. 26, 2020
(86) PCT No.: PCT/US2020/019884

§ 371 Date: Aug. 23, 2021

Related U.S. Application Data
(60) Provisional application No. 62/812,002, filed on Feb. 28, 2019.

(51) Int. Cl. A61K 31/506; A61K 45/06; A61P 35/00

(52) U.S. Cl. CPC A61K 31/506; A61P 35/00; A61K 45/06

(57) ABSTRACT

One aspect described herein includes a method for treating a multiple myeloma (MM) in a subject in need thereof comprising, administering to the subject an effective amount of a small molecule compound. More particularly, another aspect described herein includes a method for treating a multiple myeloma in a subject in need thereof comprising, administering to the subject an effective amount of the small molecule compound described herein in combination with a chemotherapeutic agent.

### CROSS-REFERENCE TO RELATED APPLICATIONS

This application claims priority to U.S. Provisional Application No. 62/812,002, filed on 28 Feb. 2019. The contents of that application are incorporated by reference herein.

### FIELD

Described herein is a method for treating a myeloma in a subject in need thereof comprising, administering to the subject an effective amount of a small molecule compound. More particularly described herein is a method for treating a multiple myeloma (MM) in a subject in need thereof comprising, administering to the subject an effective amount of a small molecule compound alone or in combination with a chemotherapeutic agent.

### BACKGROUND

Irrespective of the remarkable progress throughout the last decade, multiple myeloma (MM) remains a disease that is difficult to treat, particularly in the relapsed/refractory setting. While some chemotherapeutics are available to certain patients, MM remains a cancer that is difficult to treat, particularly when proliferation for a relapsed or refractory cancer is triggered again later. The polycomb group protein BMI-1 represents a prominent example as one of the factors that triggers proliferation for a relapsed or refractory cancer. Initially linked to the pathogenesis of MM more than a decade ago, with close associations to high-risk genes such as MYC and FOXM1, targeting of BMI-1 is still hindered by the lack of clinically effective compounds. Accordingly, there remains an urgent need to identify clinically effective therapeutic agents for use in treating MM.

### SUMMARY

One aspect described herein is the use of a small molecule Compound 1, having the name 5-fluoro-2-(6-fluoro-2-methyl-1H-benzo[d]imidazol-1-yl)-N⁴-[4-(trifluoromethyl)phenyl]pyrimidine-4,6-diamine and the structure of Formula (1):

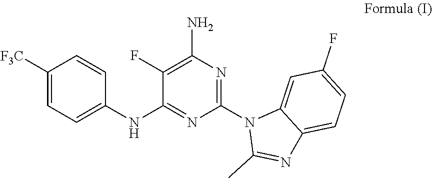 [SMILES: Cc1nc2ccc(F)cc2n1-c1nc(N)c(F)c(Nc2ccc(C(F)(F)F)cc2)n1]

or a pharmaceutically acceptable salt or pharmaceutical composition thereof in treating a myeloma.

One aspect described herein is a method for treating a multiple myeloma (MM) in a subject in need thereof comprising, administering to the subject an effective amount of Compound 1.

Another aspect described herein is a method for treating MM in a subject in need thereof comprising, administering to the subject an effective amount of Compound 1 in combination with an effective amount of one or more chemotherapeutic agents.

One aspect described herein is a use of Compound 1 in preparing a medicament for use in treating MM in a subject in need thereof comprising, administering to the subject an effective amount of the medicament.

Another aspect described herein is a use of Compound 1 in preparing a medicament for use in treating MM in a subject in need thereof comprising, administering to the subject an effective amount of the medicament in combination with a chemotherapeutic agent.

### BRIEF DESCRIPTION OF THE DRAWINGS

FIG. 1. Shows a dose response reduction in bone marrow (BM) infiltration of proliferating plasma cells after treatment with Compound 1 in comparison to vehicle (*P<0.0001).

FIG. 2. Shows the effect on hemoglobin level after treatment with Compound 1 at various dose levels in comparison to vehicle.

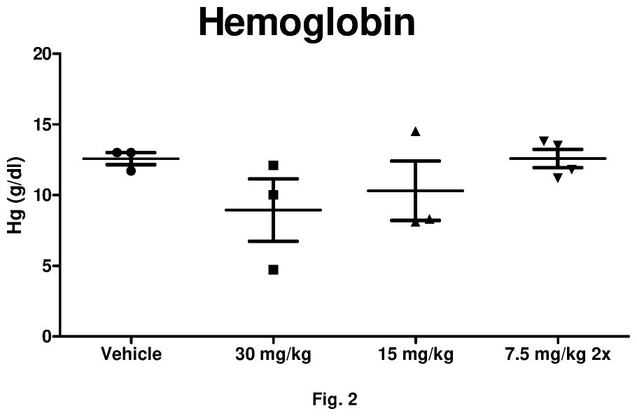

FIG. 3. Shows the effect on platelet count after treatment with Compound 1 at various dose levels in comparison to vehicle.

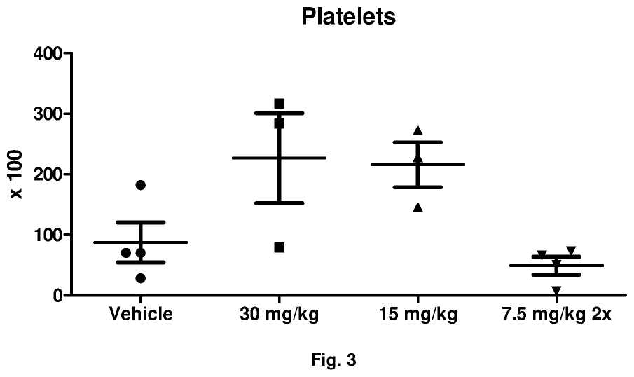

FIG. 4. Shows the effect on white blood cell (WBC) count platelet count after treatment with Compound 1 at various dose levels in comparison to vehicle.

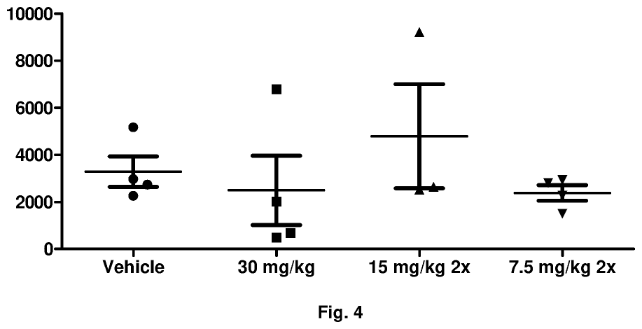

FIG. 5. Shows the effect on neutrophil count after treatment with Compound 1 at various dose levels in comparison to vehicle.

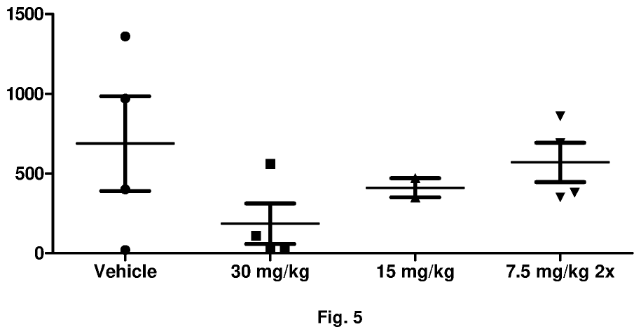

### DETAILED DESCRIPTION

One aspect described herein is the use of a small molecule Compound 1, having the name 5-fluoro-2-(6-fluoro-2-methyl-1H-benzo[d]imidazol-1-yl)-N⁴-[4-(trifluoromethyl)phenyl]pyrimidine-4,6-diamine and the structure of Formula (1):

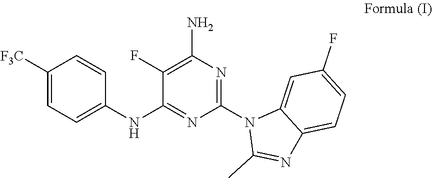 [SMILES: Cc1nc2ccc(F)cc2n1-c1nc(N)c(F)c(Nc2ccc(C(F)(F)F)cc2)n1]

or a pharmaceutically acceptable salt or pharmaceutical composition thereof in treating a myeloma. Compound 1 and a method for making the same are disclosed in International Publication Number WO2014/081906 (cited as compound 109).

One aspect described herein is a method for treating a multiple myeloma (MM) in a subject in need thereof comprising, administering to the subject an effective amount of Compound 1.

Another aspect described herein is a method for treating MM in a subject in need thereof comprising, administering to the subject an effective amount of Compound 1 in combination with an effective amount of one or more chemotherapeutic agents.

One aspect described herein is a use of Compound 1 in preparing a medicament for use in treating MM in a subject in need thereof comprising, administering to the subject an effective amount of the medicament.

Another aspect described herein is a use of Compound 1 in preparing a medicament for use in treating MM in a subject in need thereof comprising, administering to the subject an effective amount of the medicament in combination with a chemotherapeutic agent.

### Definitions

As used herein, the term “about” means a range around a given value wherein the resulting value is substantially the same as the expressly recited value. In one aspect, “about” means within 25% of a given value or range. For example, the phrase “about 70% by weight” comprises at least all values from 52% to 88% by weight. In another aspect, the term “about” means within 10% of a given value or range. For example, the phrase “about 70% by weight” comprises at least all values from 63% to 77% by weight. In another aspect, the term “about” means within 7% of a given value or range. For example, the phrase “about 70% by weight” comprises at least all values from 65% to 75% by weight. Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range was explicitly recited.

As used herein, the terms “therapies” and “therapy” can refer to any protocol(s), method(s), compositions, formulations, and/or agent(s) that can be used in the prevention, treatment, management, or amelioration of a condition or disorder or one or more symptoms thereof (e.g., a multiple myeloma or one or more symptoms or one or more conditions associated therewith).

In certain aspects, the terms “therapies” and “therapy” refer to drug therapy such as chemotherapy, adjuvant therapy, radiation, surgery, biological therapy, supportive therapy, antiviral therapy and/or other therapies useful in treatment, management, prevention, or amelioration of a condition or disorder or one or more symptoms thereof (e.g., a multiple myeloma or one or more symptoms or one or more conditions associated therewith). In certain aspects, the term “therapy” refers to a therapy other than Compound 1 or a pharmaceutically acceptable salt or pharmaceutical composition thereof. In specific aspects, an “additional therapy” and “additional therapies” refer to a therapy other than a treatment using Compound 1 or a pharmaceutically acceptable salt or pharmaceutical composition thereof. In a specific aspect, a therapy includes the use of Compound 1 as an adjuvant therapy. For example, using Compound 1 in conjunction with a drug therapy such as chemotherapy, biological therapy, surgery, supportive therapy, antiviral therapy and/or other therapies useful in treatment, management, prevention, or amelioration of a condition or disorder or one or more symptoms thereof (e.g., a multiple myeloma or one or more symptoms or one or more conditions associated therewith).

As used herein, the term “subject” refers to an individual being administered a therapy as described herein. In a specific aspect, the individual is a human.

As used herein, the term “multiple myeloma” refers to a multiple myeloma generally as described herein. In a specific aspect, the general term “myeloma” may also be used to refer to multiple myeloma without specifically using the term multiple myeloma.

As used herein, the term “effective amount” in the context of administering Compound 1 to a subject having a multiple myeloma refers to the dose of Compound 1 that results in a beneficial or therapeutic effect. In specific aspects, an “effective amount” of Compound 1 refers to an amount of Compound 1 which is sufficient to achieve at least one, two, three, four or more of the following beneficial or therapeutic effects: (i) inhibition of a multiple myeloma; (ii) regression of the multiple myeloma; (iii) eradication, removal, or complete remission of the multiple myeloma; (iv) prevention of the development or onset of one or more symptoms associated with the multiple myeloma; (v) reduction or amelioration of the severity of one or more symptoms associated with the multiple myeloma; (vi) the reduction in the number of one or more symptoms associated with the multiple myeloma; (vii) amelioration of the severity of one or more symptoms associated with the multiple myeloma; (viii) reduction in the duration of one or more symptoms associated with the multiple myeloma; (ix) prevention in the recurrence of proliferation or one or more symptoms associated with the multiple myeloma; (x) a reduction in mortality; (xi) an increase in survival rate of subjects; (xii) an increase in relapse free survival; (xiii) an increase in the number of multiple myeloma subjects in remission; (xiv) reduction in hospitalization of a subject; (xv) reduction in hospitalization length; (xvi) a decrease in hospitalization rate; (xvii) an increase in the survival of a subject; (xviii) an increase in symptom-free survival of a multiple myeloma subject; (xix) an increase in the length of a period of remission of a multiple myeloma in a subject; (xx) improvement in quality of life (QOL) as assessed by methods well known in the art, e.g., QOL questionnaires and the like; (xxi) a reduction in proliferation from administration of Compound 1 before treatment with another chemotherapeutic agent; (xxii) a reduction in proliferation from administration of Compound 1 after treatment with another chemotherapeutic agent; (xxiii) a reduction in proliferation in a combination therapy from administration of Compound 1 with another chemotherapeutic agent; (xxiv) an additive antiproliferative effect in a combination therapy from administration of Compound 1 with another chemotherapeutic agent; (xxv) a synergistic antiproliferative effect in a combination therapy from administration of Compound 1 with another chemotherapeutic agent; (xxvi) a reduction in proliferation from administration of Compound 1 before therapy with radiation; (xxvii) a reduction in proliferation from administration of Compound 1 after therapy with radiation; (xxviii) a reduction in proliferation from administration of Compound 1 in a combination therapy with radiation; (xxix) a reduction in proliferation from administration of Compound 1 before treatment with surgery; (xxx) a reduction in proliferation from administration of Compound 1 in a combination treatment with surgery; (xxxi) enhancement of or improvement of the therapeutic effect from administration of Compound 1 with a palliative therapy; (xxxii) a decrease in the plasma concentration of BMI-1 in a subject having a multiple myeloma; (xxxiii) a decrease in circulating proliferative cells in the plasma of a subject having a multiple myeloma; (xxxiv) an alteration (e.g., a decrease or increase) in the plasma concentration of a multiple myeloma biomarker in a subject having a multiple myeloma (e.g., BMI-1, tubulin polymerization, apoptotic markers or tissue and the like); (xxxv) reduction in the concentration of BMI-1 in a biological specimen (e.g., plasma, serum, urine, or any other biofluids) from a subject having a multiple myeloma; (xxxvi) proliferative cell count is reduced after administration of a therapy as described herein as measured by conventional methods available to one skilled in the art, such as magnetic resonance imaging (MRI), dynamic contrast-enhanced MRI (DCE-MRI), X-ray, computed tomography (CT) scan, positron emission tomography (PET) scan, 7-AAD fluorescence, or DAPI fluorescence; (xxxvii) proliferative cell count is maintained after administration of a therapy as described herein as measured by conventional methods available to one skilled in the art, such as magnetic resonance imaging (MRI), dynamic contrast-enhanced MRI (DCE-MRI), X-ray, computed tomography (CT) scan, positron emission tomography (PET) scan, 7-AAD fluorescence, or DAPI fluorescence; or, (xxxviii) proliferative cell count does not increase or increases by less than expected after administration of a therapy as described herein as measured by conventional methods available to one skilled in the art, such as magnetic resonance imaging (MRI), dynamic contrast-enhanced MRI (DCE-MRI), X-ray, computed tomography (CT) scan, or a positron emission tomography (PET) scan, 7-AAD fluorescence, or DAPI fluorescence.

As used herein, the term “in a 24 hour period” refers to a period of time over which a condition is maintained; for example, the effective amount of Compound 1 is identified when the mean plasma concentration of Compound 1 is achieved and maintained for a plurality of 24 hour periods. In other words, the mean plasma concentration of Compound 1 may be reached in a suitable time, which may be more or less than 24 hours.

As used herein, the term “a therapy as described herein” refers to a method of use for Compound 1 or a pharmaceutically acceptable salt or pharmaceutical composition thereof for use in treating or ameliorating a multiple myeloma in a subject in need thereof comprising, administering to the subject an effective amount of Compound 1.

In one aspect of the therapy described herein, the use or method of use of Compound 1 includes a pharmaceutically acceptable salt or pharmaceutical composition thereof. In another aspect of the therapy described herein, the use or method of use of Compound 1 includes the use or method of use of Compound 1, a pharmaceutically acceptable salt or pharmaceutical composition of Compound 1, or a combination of Compound 1 or a pharmaceutically acceptable salt or pharmaceutical composition thereof with another chemotherapeutic agent(s), wherein the combination has synergistic antiproliferative activity. In another aspect, the other chemotherapeutic agent inhibits tubulin polymerization. In another aspect, the other chemotherapeutic agent inhibits BMI-1 functional activity.

As used herein, the term “pharmaceutically acceptable salt(s)” refers to a salt prepared from a pharmaceutically acceptable non-toxic acid or base including an inorganic acid and base and an organic acid and base; see, for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein, the term “Compound 1” refers to 5-fluoro-2-(6-fluoro-2-methyl-1H-benzo[d]imidazol-1-yl)-N⁴-[4-(trifluoromethyl)phenyl]pyrimidine-4,6-diamine or a pharmaceutically acceptable salt or pharmaceutical composition thereof. In various aspects, the term “Compound 1” refers to Compound 109 disclosed in International Publication No. WO2014/081906, which is incorporated in its entirety by reference herein.

### Method of Use

Without being limited by theory, mechanistic studies have demonstrated that Compound 1 inhibits microtubule polymerization, while avoiding the most debilitating toxicities of other such agents. In addition, Compound 1 combines additively or synergistically with certain standard clinical regimens, yielding potent and durable cancer regression.

As demonstrated herein, Compound 1 or a pharmaceutically acceptable salt or pharmaceutical composition thereof is a small molecule inhibitor of tubulin polymerization for use in treating or ameliorating a multiple myeloma in a subject in need thereof comprising, administering to the subject an effective amount of Compound 1 or a pharmaceutically acceptable salt or pharmaceutical composition thereof.

In one aspect of the use or method of use described herein, the use or method of use of Compound 1 includes a pharmaceutically acceptable salt or pharmaceutical composition thereof. In another aspect of the use or method of use described herein, the use or method of use of Compound 1 includes the use or method of use of Compound 1, the use or method of use of a pharmaceutically acceptable salt or pharmaceutical composition of Compound 1, or the use or method of use of a combination of Compound 1 or a pharmaceutically acceptable salt or pharmaceutical composition thereof with another chemotherapeutic agent(s), wherein the combination has additive or synergistic antiproliferative activity. In another aspect, the other chemotherapeutic agent inhibits tubulin polymerization. In another aspect, the other chemotherapeutic agent inhibits BMI-1 functional activity.

In one aspect, methods for inhibiting or reducing tubulin polymerization, which methods may also indirectly inhibit BMI-1 function to induce cell-cycle arrest in a proliferating cell or cell line are described herein.

In another aspect, a method for inhibiting or reducing tubulin polymerization and indirectly inhibiting BMI-1 function to induce cell-cycle arrest in a proliferating cell or cell line comprises, contacting Compound 1 or a pharmaceutically acceptable salt or pharmaceutical composition thereof with a proliferating cell or cell line, which proliferating cell or cell line may be naïve or has been shown to be affected by the inhibition or a reduction of tubulin polymerization and BMI-1 function.

In another aspect, non-limiting examples of such cells or cell lines are selected from HL-60, HeLa, HT1080, HCT116, HEK293, NCI H460, U-87MG, ASPC-1, PL-45, HPAF-2, PC-3, MDA-MB-231, MDA-MB-468, A431, SNU-1, AGS, Kato III, A549, Calu-6, A375, SY5Y, SKOV3, Capan-1, sNF96.2, TIVE-L1, TIVE-L2, LNCaP cells and the like. In a more specific aspect, the cell or cell line may be a multiple myeloma cell.

In one aspect, a method for inhibiting or reducing tubulin polymerization and BMI-1 function in a subject having a multiple myeloma in need thereof comprises, administering an effective amount of Compound 1 or a pharmaceutically acceptable salt or pharmaceutical composition thereof to the subject as described herein.

In a specific aspect, the subject diagnosed with a multiple myeloma is capable of being treated by a chemotherapeutic agent for inhibiting or reducing tubulin polymerization.

In a specific aspect, the subject diagnosed with a multiple myeloma is capable of being treated by a chemotherapeutic agent for inhibiting or reducing BMI-1 function.

In a specific aspect, a method for inhibiting or reducing tubulin polymerization as described herein inhibits or reduces tubulin polymerization by about 5%, 10%, 15%, 20%, 25%, 30%, 35%, 40%, 45%, 50%, 55%, 60%, 65%, 80%, 85%, 90%, 95%, or 100% relative to tubulin polymerization prior to administration of Compound 1 to the subject, as assessed by methods well known in the art.

In a specific aspect, a method for inhibiting or reducing BMI-1 function as described herein inhibits BMI-1 function by about 5%, 10%, 15%, 20%, 25%, 30%, 35%, 40%, 45%, 50%, 55%, 60%, 65%, 80%, 85%, 90%, 95%, or 100% relative to BMI-1 function prior to administration of Compound 1 to the subject, as assessed by methods well known in the art.

In a specific aspect, a method for inhibiting or reducing tubulin polymerization as described herein inhibits or reduces tubulin polymerization in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to tubulin polymerization prior to administration of Compound 1 to the subject, as assessed by methods well known in the art.

In a specific aspect, a method for inhibiting or reducing BMI-1 function as described herein inhibits or reduces BMI-1 function in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to BMI-1 function prior to administration of Compound 1 to the subject, as assessed by methods well known in the art.

In a specific aspect, a method for inhibiting or reducing tubulin polymerization as described herein inhibits proliferation or reduces an in vitro or in vivo proliferating cell or cell line population by about 5%, 10%, 15%, 20%, 25%, 30%, 35%, 40%, 45%, 50%, 55%, 60%, 65%, 80%, 85%, 90%, 95%, or 100%, relative to the in vitro or in vivo proliferating cell or cell line population prior to administration of Compound 1 to the subject, as assessed by methods well known in the art.

In a specific aspect, a method for inhibiting or reducing BMI-1 function as described herein inhibits proliferation or reduces an in vitro or in vivo proliferating cell or cell line population by about 5%, 10%, 15%, 20%, 25%, 30%, 35%, 40%, 45%, 50%, 55%, 60%, 65%, 80%, 85%, 90%, 95%, or 100%, relative to the in vitro or in vivo proliferating cell or cell line population prior to administration of Compound 1 to the subject, as assessed by methods well known in the art.

In a specific aspect, a method for inhibiting or reducing tubulin polymerization as described herein inhibits proliferation or reduces an in vitro or in vivo proliferating cell or cell line population in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to the in vitro or in vivo proliferating cell or cell line population prior to administration of Compound 1 to the subject, as assessed by methods well known in the art.

In a specific aspect, a method for inhibiting or reducing BMI-1 function as described herein inhibits proliferation or reduces an in vitro or in vivo proliferating cell or cell line population in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to the in vitro or in vivo proliferating cell or cell line population prior to administration of Compound 1 to the subject, as assessed by methods well known in the art.

In various aspects, a method for inhibiting or reducing tubulin polymerization as described herein reduces the expression of GTP-bound αβ-tubulin subunits available for microtubule assembly in a subject as assessed by methods well known in the art, e.g., ELISA.

In various aspects, a method for inhibiting or reducing BMI-1 function as described herein reduces the plasma concentration of BMI-1 in a subject as assessed by methods well known in the art, e.g., ELISA.

In one aspect, a method for preventing, treating or ameliorating a multiple myeloma in a subject in need thereof comprises, administering an amount of Compound 1 effective to inhibit or reduce tubulin polymerization in the subject is described herein.

In one aspect, a method for preventing, treating or ameliorating a multiple myeloma in a subject in need thereof comprises, administering an amount of Compound 1 effective to inhibit or reduce BMI-1 function in the subject is described herein.

In a specific aspect, a method for preventing, treating or ameliorating a multiple myeloma in a subject in need thereof as described herein inhibits or reduces tubulin polymerization by about 5%, 10%, 15%, 20%, 25%, 30%, 35%, 40%, 45%, 50%, 55%, 60%, 65%, 80%, 85%, 90%, 95%, or 100% relative to tubulin polymerization prior to administration of Compound 1 to the subject, as assessed by methods well known in the art.

In a specific aspect, a method for preventing, treating or ameliorating a multiple myeloma in a subject in need thereof as described herein inhibits or reduces BMI-1 function by about 5%, 10%, 15%, 20%, 25%, 30%, 35%, 40%, 45%, 50%, 55%, 60%, 65%, 80%, 85%, 90%, 95%, or 100% relative to BMI-1 function prior to administration of Compound 1 to the subject, as assessed by methods well known in the art.

In a specific aspect, a method for preventing, treating or ameliorating a multiple myeloma in a subject in need thereof as described herein inhibits or reduces tubulin polymerization in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to tubulin polymerization prior to administration of Compound 1 to the subject, as assessed by methods well known in the art.

In a specific aspect, a method for preventing, treating or ameliorating a multiple myeloma in a subject in need thereof as described herein inhibits or reduces BMI-1 function in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to BMI-1 function prior to administration of Compound 1 to the subject, as assessed by methods well known in the art.

In various aspects, a method for preventing, treating or ameliorating a multiple myeloma in a subject in need thereof as described herein reduces the concentration of BMI-1 in a subject as assessed by methods well known in the art, e.g., ELISA.

In one aspect, a method for preventing, treating or ameliorating a multiple myeloma in a subject in need thereof comprises, administering an amount of Compound 1 effective to inhibit proliferation or reduce an in vitro or in vivo proliferating cell or cell line population in the subject is described herein.

In a specific aspect, a method for preventing, treating or ameliorating a multiple myeloma in a subject in need thereof as described herein inhibits proliferation or reduces an in vitro or in vivo proliferating cell or cell line population in the subject by about 5%, 10%, 15%, 20%, 25%, 30%, 35%, 40%, 45%, 50%, 55%, 60%, 65%, 80%, 85%, 90%, 95%, or 100% relative to proliferation or in vitro or in vivo proliferating cell or cell line population in the subject prior to administration of Compound 1 to the subject, as assessed by methods well known in the art.

In a specific aspect, a method for preventing, treating or ameliorating a multiple myeloma in a subject in need thereof as described herein inhibits proliferation or reduces an in vitro or in vivo proliferating cell or cell line population in the subject in a range of from about 5% to about 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, or from about 40% to about 100%, or any range in between, relative to proliferation or in vitro or in vivo proliferating cell or cell line population in the subject prior to administration of Compound 1 to the subject, as assessed by methods well known in the art.

In various aspects, a method for preventing, treating or ameliorating a multiple myeloma in a subject in need thereof as described herein inhibits proliferation or reduces an in vitro or in vivo proliferating cell or cell line population in a subject as assessed by methods well known in the art, e.g., ELISA.

In one aspect, a method for preventing, treating or ameliorating a multiple myeloma in a subject in need thereof comprises, administering an amount of Compound 1 effective to inhibit proliferation or reduce an in vitro or in vivo proliferating cell or cell line population in the subject in combination with another therapy (e.g., one or more additional therapies that do not comprise Compound 1, or that comprise a different anti-proliferative agent) to a subject in need thereof is described herein.

Such methods may involve administering Compound 1 prior to, concurrent with, or subsequent to administration of the additional therapy. In certain aspects, such methods have an additive or synergistic effect.

In a specific aspect, presented herein is a method for preventing, treating or ameliorating a multiple myeloma in a subject in need thereof comprising, administering to a subject in need thereof an effective amount of Compound 1 and an effective amount of another therapy.

One aspect described herein includes a hematologic cancer that can be prevented, treated or ameliorated in accordance with the methods provided herein include, but are not limited to, a multiple myeloma.

In one aspect, presented herein is a method for preventing, treating or ameliorating a multiple myeloma, comprising: (a) administering to a subject in need thereof one or more doses of Compound 1 or a pharmaceutically acceptable salt or pharmaceutical composition thereof a pharmaceutical composition thereof; and (b) monitoring the concentration of certain biomarkers, before and/or after step (a).

In a specific aspect, the monitoring step (b) is carried out before and/or after a certain number of doses (e.g., 1, 2, 4, 6, 8, 10, 12, 14, 15, or 29 doses, or more doses; 2 to 4, 2 to 8, 2 to 20 or 2 to 30 doses) or a certain time period (e.g., 1, 2, 3, 4, 5, 6, or 7 days; or 1, 2, 3, 4, 5, 10, 15, 20, 30, 40, 45, 48, or 50 weeks) of administering Compound 1 or a pharmaceutically acceptable salt or pharmaceutical composition thereof.

In a specific aspect, one or more of these monitoring parameters are detected prior to administration of Compound 1 or a pharmaceutically acceptable salt or pharmaceutical composition thereof to the subject.

In a specific aspect, a decrease in the proliferation of an in vitro or in vivo proliferating cell or cell line population following administration of Compound 1 or a pharmaceutically acceptable salt or pharmaceutical composition thereof indicates that the course of treatment is effective for preventing, treating or ameliorating the multiple myeloma.

a specific aspect, a change in the proliferation of an in vitro or in vivo proliferating cell or cell line population following administration of Compound 1 or a pharmaceutically acceptable salt or pharmaceutical composition thereof may indicate that the dosage, frequency and/or length of administration of Compound 1 or a pharmaceutically acceptable salt or pharmaceutical composition thereof may be adjusted (e.g., increased, reduced or maintained).

In a specific aspect, the concentration of certain biomarkers in biological specimens of a subject is monitored before, during and/or after a course of treatment for a multiple myeloma involving the administration of Compound 1 or a pharmaceutically acceptable salt or pharmaceutical composition thereof to the subject.

The dosage, frequency and/or length of administration of Compound 1 or a pharmaceutically acceptable salt or pharmaceutical composition thereof to a subject might be modified as a result of the proliferation of an in vitro or in vivo proliferating cell or cell line population. Alternatively, the changes in these monitoring parameters (e.g., concentration of certain biomarkers) might indicate that the course of treatment involving the administration of the Compound 1 or a pharmaceutically acceptable salt or pharmaceutical composition thereof is effective in preventing, treating or ameliorating the multiple myeloma.

The concentration of certain biomarkers in a subject may be detected by any technique known to one of skill in the art. In certain aspects, the method for detecting the concentration of certain biomarkers of a subject comprises obtaining a biological sample (e.g., tissue or fluid sample) from the subject and detecting the concentration of the biomarkers in the biological sample (e.g., from plasma, serum, urine, or any other biofluids), that has been subjected to certain types of treatment (e.g., centrifugation), and detection by use of immunological techniques, such as ELISA.

In a specific aspect, an ELISA assay, as described herein, may be used to detect the concentration of the biomarkers in a biological sample (e.g., from plasma, serum, urine, or any other biofluids) that has been subjected to certain types of treatment (e.g., centrifugation). Other techniques known in the art that may be used to detect the concentration of the biomarkers in a biological sample include multiplex or proteomic assays.

In specific aspects, the methods for preventing, treating or ameliorating a multiple myeloma provided herein alleviate or manage one, two or more symptoms associated with the multiple myeloma. Alleviating or managing one, two or more symptoms of the multiple myeloma may be used as a clinical endpoint for efficacy of Compound 1 or a pharmaceutically acceptable salt or pharmaceutical composition thereof for preventing, treating or ameliorating the multiple myeloma. In some aspects, the methods for preventing, treating or ameliorating the multiple myeloma provided herein reduce the duration and/or severity of one or more symptoms associated with the multiple myeloma. In some aspects, the methods for preventing, treating or ameliorating the multiple myeloma provided herein inhibit the onset, progression and/or recurrence of one or more symptoms associated with the multiple myeloma. In some aspects, the methods for treating the multiple myeloma provided herein reduce the number of symptoms associated with the multiple myeloma.

In certain aspects, the methods for preventing, treating or ameliorating a multiple myeloma provided herein prolong or delay the G1/S or late G1/S phase of the cell cycle (i.e., the period between the late checkpoint (resting or pre-DNA synthesis phase), and the early DNA synthesis phase). In other aspects, the methods for preventing, treating or ameliorating a multiple myeloma provided herein prolong or delay the S or G2/M phase of the cell cycle (i.e., the period between DNA synthesis and the early division phase).

In some aspects, the methods for preventing, treating or ameliorating a multiple myeloma provided herein reduce, ameliorate, or alleviate the severity of the multiple myeloma and/or one or more symptoms thereof. In other aspects, the methods for preventing, treating or ameliorating a multiple myeloma provided herein reduce hospitalization (e.g., the frequency or duration of hospitalization) of a subject diagnosed with the multiple myeloma.

In certain aspects, the methods provided herein increase the survival of a subject diagnosed with a multiple myeloma. In specific aspects, the methods provided herein increase the survival of a subject diagnosed with a multiple myeloma by about 6 months or more, about 7 months or more, about 8 months or more, about 9 months or more, or about 12 months or more.

In particular aspects, the methods for preventing, treating or ameliorating a multiple myeloma provided herein inhibit or reduce the progression of the multiple myeloma, or one or more symptoms associated therewith. In specific aspects, the methods for preventing, treating or ameliorating a multiple myeloma provided herein enhance or improve the therapeutic effect of another therapy (e.g., an anti-cancer agent, radiation, drug therapy, such as chemotherapy, anti-androgen therapy, or surgery). In certain aspects, the methods for preventing, treating or ameliorating a multiple myeloma provided herein involve the use of Compound 1 or a pharmaceutically acceptable salt or pharmaceutical composition thereof as an adjuvant therapy.

In particular aspects, the methods for preventing, treating or ameliorating a multiple myeloma provided herein reduce the mortality of subjects diagnosed with the multiple myeloma. In certain aspects, the methods for preventing, treating or ameliorating a multiple myeloma provided herein increase the number of subjects in remission or decrease the hospitalization rate. In other aspects, the methods for preventing, treating or ameliorating a multiple myeloma provided herein prevent the development, onset or progression of one or more symptoms associated with the multiple myeloma.

In particular aspects, the methods for preventing, treating or ameliorating a multiple myeloma provided herein increase symptom-free survival of multiple myeloma subjects. In some aspects, the methods for preventing, treating or ameliorating a multiple myeloma provided herein do not cure the multiple myeloma in subjects, but prevent the progression or worsening of the disease. In some aspects, the methods for preventing, treating or ameliorating a multiple myeloma provided herein improve the subject's quality of life.

In certain aspects, the methods for preventing, treating or ameliorating a multiple myeloma provided herein increase the cancer-free survival rate of subjects diagnosed with the cancer. In some aspects, the methods for preventing, treating or ameliorating a multiple myeloma provided herein increase relapse-free survival. In certain aspects, the methods for preventing, treating or ameliorating a multiple myeloma provided herein increase the number of subjects in remission. In other aspects, the methods for preventing, treating or ameliorating a multiple myeloma provided herein increase the length of remission in subjects.

### Treatment Population

In one aspect, a subject treated for a multiple myeloma in accordance with the methods provided herein is a human who has or is diagnosed with a multiple myeloma. In another aspect, a subject treated for a multiple myeloma in accordance with the methods provided herein is a human predisposed or susceptible to a multiple myeloma. In another aspect, a subject treated for a multiple myeloma in accordance with the methods provided herein is a human at risk of developing a multiple myeloma. In another aspect, a subject treated for a multiple myeloma in accordance with the methods provided herein is a human having a genetic or somatic mutation placing the subject at risk or predisposition for developing a multiple myeloma.

In one aspect, a subject treated for a multiple myeloma in accordance with the methods provided herein is a human infant. In another aspect, a subject treated for a multiple myeloma in accordance with the methods provided herein is a human toddler. In another aspect, a subject treated for a multiple myeloma in accordance with the methods provided herein is a human child. In another aspect, a subject treated for a multiple myeloma in accordance with the methods provided herein is a human adult. In another aspect, a subject treated for a multiple myeloma in accordance with the methods provided herein is a middle-aged human. In another aspect, a subject treated for a multiple myeloma in accordance with the methods provided herein is an elderly human.

In certain aspects, a subject treated for cancer in accordance with the methods provided herein has a multiple myeloma metastasized to other areas of the body, such as the bones, lung and liver. In certain aspects, a subject treated for multiple myeloma in accordance with the methods provided herein is in remission from the multiple myeloma. In some aspects, the subject treated for multiple myeloma in accordance with the methods provided herein had a recurrence of the multiple myeloma. In certain aspects, a subject treated in accordance with the methods provided herein is experiencing recurrence of one or more symptoms associated with the multiple myeloma.

In certain aspects, a subject treated for a multiple myeloma in accordance with the methods provided herein is i). a human toddler that is in an age range of from about 1 to about 5 years old; ii). a human child that is in an age range of from about 5 to 10 years old; or, from about 10 to about 18 years old; ii). a human adult that is in an age range of from about 18 to about 30 years old; or, from about 25 to about 35 years old; or, from about 35 to about 45 years old ii). a middle-aged human adult that is in an age range of from about 40 to about 55 years old; or, from about 50 to about 65 years old ii). a human adult that is in an age range of from about 60 to about 75 years old, ii). a human toddler that is about 70 to about 85 years old, about 80 to about 90 years old, about 90 to about 95 years old or about 95 to about 100 years old, or any age in between.

In a specific aspect, a subject treated for a multiple myeloma in accordance with the methods provided herein is a human that is 18 years old or older. In a particular aspect, a subject treated for a multiple myeloma in accordance with the methods provided herein is a human child that is between the age of 1 year old to 18 years old. In a certain aspect, a subject treated for a multiple myeloma in accordance with the methods provided herein is a human that is between the age of 12 years old and 18 years old. In a certain aspect, the subject is a male human. In another aspect, the subject is a female human. In one aspect, the subject is a female human that is not pregnant or is not breastfeeding. In one aspect, the subject is a female that is pregnant or will/might become pregnant, or is breast feeding.

As used herein, the term “human infant” refers to a newborn to 1 year old human.

As used herein, the term “human toddler” refers to a human that is 1 year to 5 years old.

As used herein, the term “human child” refers to a human that is 5 years to 18 years old.

As used herein, the term “human adult” refers to a human that is 18 years or older.

As used herein, the term “middle-aged human” refers to a human between the ages of 40 and 65.

As used herein, the term “elderly human” refers to a human 65 years or older.

In particular aspects, a subject treated for a multiple myeloma in accordance with the methods provided herein is a human that is in an immunocompromised state or immunosuppressed state. In certain aspects, a subject treated for a multiple myeloma in accordance with the methods provided herein is a human receiving or recovering from immunosuppressive therapy. In certain aspects, a subject treated for a multiple myeloma in accordance with the methods provided herein is a human that has or is at risk of getting a multiple myeloma. In certain aspects, a subject treated for a multiple myeloma in accordance with the methods provided herein is a human who is, will or has undergone surgery, drug therapy, such as chemotherapy, hormonal therapy and/or radiation therapy.

In some aspects, a subject treated for a multiple myeloma in accordance with the methods provided herein is administered Compound 1 or a pharmaceutically acceptable salt or pharmaceutical composition thereof, or a combination therapy before any adverse effects or intolerance to therapies other than Compound 1 develops. In some aspects, a subject treated for a multiple myeloma in accordance with the methods provided herein is a refractory subject. In certain aspects, a refractory subject is a subject refractory to a standard therapy (e.g., surgery, radiation and/or drug therapy such as chemotherapy). In certain aspects, a subject with a multiple myeloma is refractory to a therapy when the multiple myeloma has not significantly been eradicated and/or the one or more symptoms have not been significantly alleviated. The determination of whether a subject refractory can be made either in vivo or in vitro by any method known in the art for assaying the effectiveness of a treatment of a multiple myeloma, using art-accepted meanings of “refractory” in such a context.

In some aspects, a subject treated for a multiple myeloma in accordance with the methods provided herein is a human that has proven refractory to therapies other than treatment with Compound 1 or a pharmaceutically acceptable salt or pharmaceutical composition thereof, but is no longer on these therapies. In certain aspects, a subject treated for a multiple myeloma in accordance with the methods provided herein is a human already receiving one or more conventional anti-cancer therapies, such as surgery, drug therapy such as chemotherapy, anti-androgen therapy or radiation. Among these subjects are refractory subjects, subjects who are too young for conventional therapies, and subjects with recurring multiple myeloma despite treatment with existing therapies.

In some aspects, a subject treated for a multiple myeloma in accordance with the methods provided herein is a human susceptible to adverse reactions to conventional therapies. In some aspects, a subject treated for a multiple myeloma in accordance with the methods provided herein is a human that has not received a therapy, e.g., drug therapy such as chemotherapy, surgery, anti-androgen therapy or radiation therapy, prior to the administration of Compound 1 or a pharmaceutically acceptable salt or pharmaceutical composition thereof. In other aspects, a subject treated for a multiple myeloma in accordance with the methods provided herein is a human that has received a therapy prior to administration of Compound 1 or a pharmaceutically acceptable salt or pharmaceutical composition thereof. In some aspects, a subject treated for a multiple myeloma in accordance with the methods provided herein is a human that has experienced adverse side effects to the prior therapy or the prior therapy was discontinued due to unacceptable levels of toxicity to the human.

### Dosage and Administration

In accordance with the methods for preventing, treating or ameliorating a multiple myeloma provided herein, Compound 1 or a pharmaceutically acceptable salt or pharmaceutical composition thereof can be administered to a subject in need thereof by a variety of routes in amounts which result in a beneficial or therapeutic effect. Compound 1 or a pharmaceutically acceptable salt or pharmaceutical composition thereof may be orally administered to a subject in need thereof in accordance with the methods for preventing, treating or ameliorating a multiple myeloma provided herein. The oral administration of Compound 1 or a pharmaceutically acceptable salt or pharmaceutical composition thereof may facilitate subjects in need of such treatment complying with a regimen for taking Compound 1 or a pharmaceutically acceptable salt or pharmaceutical composition thereof. Thus, in a specific aspect, Compound 1 or a pharmaceutically acceptable salt or pharmaceutical composition thereof is administered orally to a subject in need thereof. In another aspect, Compound 1 or a pharmaceutically acceptable salt or pharmaceutical composition thereof provided herein can be administered orally, with or without food or water.

Other routes of administration include, but are not limited to, intravenous, intradermal, intrathecal, intramuscular, subcutaneous, intranasal, inhalation, transdermal, topical, transmucosal, intracranial, epidural and intra-synovial. In one aspect, Compound 1 or a pharmaceutically acceptable salt or pharmaceutical composition thereof is administered systemically (e.g., parenterally) to a subject in need thereof. In one aspect, Compound 1 or a pharmaceutically acceptable salt or pharmaceutical composition thereof is administered via a route that permits Compound 1 or a pharmaceutically acceptable salt or pharmaceutical composition thereof to cross the blood-brain barrier (e.g., orally).

In accordance with the methods for preventing, treating or ameliorating a multiple myeloma provided herein that involve administration of Compound 1 or a pharmaceutically acceptable salt or pharmaceutical composition thereof in combination with one or more additional therapies, Compound 1 or a pharmaceutically acceptable salt or pharmaceutical composition thereof and one or more additional therapies may be administered by the same route or a different route of administration.

The dosage and frequency of administration of Compound 1 or a pharmaceutically acceptable salt or pharmaceutical composition thereof is administered to a subject in need thereof in accordance with the methods for preventing, treating or ameliorating a multiple myeloma provided herein will be efficacious while minimizing any side effects. The exact dosage and frequency of administration of Compound 1 or a pharmaceutically acceptable salt or pharmaceutical composition thereof can be determined by a practitioner, in light of factors related to the subject that requires treatment.

Factors which may be taken into account include the severity of the disease state, general health of the subject, age, weight, and gender of the subject, diet, time and frequency of administration, drug combination(s), reaction sensitivities, and tolerance/response to therapy. The dosage and frequency of administration of Compound 1 or a pharmaceutically acceptable salt or pharmaceutical composition thereof may be adjusted over time to provide an effective amount of Compound 1 or a pharmaceutically acceptable salt or pharmaceutical composition thereof or to maintain the desired effect.

As described herein, the methods for preventing, treating or ameliorating a multiple myeloma in a subject in need thereof presented herein comprises, administering to the subject an effective amount of Compound 1 or a pharmaceutically acceptable salt or pharmaceutical composition thereof, wherein the effective amount is a dose administered to the subject twice per week on different days, wherein the second dose in a week follows the first by three days, and wherein the first dose in a following week follows the second dose in a preceding week by four days.

In a specific aspect, the effective amount is a dose administered to the subject that may be increased or decreased depending on subject response.

In a specific aspect, a method for preventing, treating or ameliorating a multiple myeloma in a subject in need thereof comprises the administration of an effective amount of Compound 1 or a pharmaceutically acceptable salt or pharmaceutical composition thereof to the subject, wherein the effective amount is a dose selected from a dose in a range of from about 50 mg to about 200 mg, from about 100 mg to about 200 mg, from about 150 mg to about 200 mg, and the like, or any range in between, administered orally twice per week.

In a specific aspect, a method for preventing, treating or ameliorating a multiple myeloma in a subject in need thereof comprises the administration of an effective amount of Compound 1 or a pharmaceutically acceptable salt or pharmaceutical composition thereof to the subject, wherein the effective amount is a dose selected from about 50 mg, about 100 mg, about 150 mg or about 200 mg, and the like, or any range in between, administered orally twice per week.

In a specific aspect, a method for preventing, treating or ameliorating a multiple myeloma in a subject in need thereof comprises the administration of an effective amount of Compound 1 or a pharmaceutically acceptable salt or pharmaceutical composition thereof to the subject, wherein the effective amount is a dose of about 50 mg administered orally twice per week.

In some aspects, a method for preventing, treating or ameliorating a multiple myeloma in a subject in need thereof comprises the administration of an effective amount of Compound 1 or a pharmaceutical composition thereof to the subject, wherein the effective amount is a dosage that is expressed as mg per meter squared (mg/m²). The mg/m² for Compound 1 may be determined, for example, by multiplying a conversion factor for an animal by an animal dose in mg per kilogram (mg/kg) to obtain the dose in mg/m² for human dose equivalent. For regulatory purposes, the following conversion factors may be used: Mouse=3, Hamster=4.1, Rat=6, Guinea Pig=7.7. (based on Freireich et al., Cancer Chemother. Rep. 50(4):219-244 (1966)). The height and weight of a human may be used to calculate a human body surface area applying Boyd's Formula of Body Surface Area. In specific aspects, a method for preventing, treating or ameliorating a multiple myeloma in a subject in need thereof comprises the administration of an effective amount of Compound 1 or a pharmaceutical composition thereof to the subject, wherein the effective amount is an amount in the range of from about 0.1 mg/m² to about 1000 mg/m², or any range in between.

In one aspect, a method for preventing, treating or ameliorating a multiple myeloma in a subject in need thereof comprises the administration of an effective amount of Compound 1 or a pharmaceutical composition thereof to the subject, wherein the effective amount is a dosage that achieves a target mean plasma concentration of Compound 1 in a subject with a multiple myeloma or an animal model with a pre-established multiple myeloma.

In a specific aspect, a method for preventing, treating or ameliorating a multiple myeloma in a subject in need thereof comprises the administration of an effective amount of Compound 1 or a pharmaceutical composition thereof to the subject, wherein the effective amount is a dosage that achieves a mean plasma concentration of Compound 1 in a 24 hour period in a range of from approximately 3 hr·μg/mL to approximately 70 hr·μg/mL, from approximately 3 hr·μg/mL to approximately 60 hr·μg/mL, from approximately 3 hr·μg/mL to approximately 50 hr·μg/mL, from approximately 3 hr·μg/mL to approximately 40 hr·μg/mL, from approximately 3 hr·μg/mL to approximately 30 hr·μg/mL, from approximately 3 hr·μg/mL to approximately 20 hr·μg/mL, from approximately 3 hr·μg/mL to approximately 10 hr·μg/mL, and the like, or any range in between, in a subject with the multiple myeloma or an animal model with a pre-established multiple myeloma.

In a specific aspect, a method for preventing, treating or ameliorating a multiple myeloma in a subject in need thereof comprises the administration of an effective amount of Compound 1 or a pharmaceutical composition thereof to the subject, wherein the effective amount is a dosage that achieves a mean plasma concentration of Compound 1 in a 24 hour period of approximately 3 hr·μg/mL, approximately 10 hr·μg/mL, approximately 20 hr·μg/mL, approximately 30 hr·μg/mL, approximately 40 hr·μg/mL, approximately 50 hr·μg/mL, approximately 60 hr·μg/mL, approximately 70 hr·μg/mL, and the like, or any range in between, in a subject with the multiple myeloma or an animal model with a pre-established multiple myeloma.

To achieve such plasma concentrations, a dose described herein of Compound 1 or a pharmaceutical composition thereof may be administered. In certain aspects, subsequent doses of Compound 1 or a pharmaceutical composition thereof may be adjusted accordingly based on the mean plasma concentrations of Compound 1 achieved with a dose of Compound 1 or a pharmaceutical composition thereof administered to the subject.

In specific aspects, a method for preventing, treating or ameliorating a multiple myeloma in a subject in need thereof comprises the administration of an effective amount of Compound 1 or a pharmaceutical composition thereof to the subject, wherein the effective amount is a dosage that achieves a reduced target mean plasma concentration of one or more biomarkers in a subject with the multiple myeloma or an animal model with a pre-established multiple myeloma.

In particular aspects, a method for preventing, treating or ameliorating a multiple myeloma in a subject in need thereof comprises the administration of an effective amount of Compound 1 or a pharmaceutical composition thereof to the subject, wherein the effective amount is a dosage that achieves the desired tissue to mean plasma concentration ratios of Compound 1 or a pharmaceutical composition thereof as determined, e.g., by any imaging techniques known in the art, in a subject with the multiple myeloma or an animal model with a pre-established multiple myeloma.

In some aspects, a method for preventing, treating or ameliorating a multiple myeloma in a subject in need thereof comprises the administration of an effective amount of Compound 1 or a pharmaceutical composition thereof to the subject, wherein the effective amount may or may not be the same for each dose. In particular aspects, a first (i.e., initial) dose of Compound 1 or a pharmaceutical composition thereof is administered to a subject in need thereof for a first period of time, followed by a second (i.e., loading) dose of Compound 1 or a pharmaceutical composition thereof is administered to the subject for a second period of time and, subsequently, a third (i.e., maintenance) dose of Compound 1 or a pharmaceutical composition thereof is administered to the subject for a second period of time. The first dose may be more than the second dose, or the first dose may be less than the second dose. In similar fashion, the third dose of Compound 1 or a pharmaceutical composition thereof may be more or less than the second dose and more or less than the first dose.

In some aspects, the dosage amounts described herein refer to total amounts administered; that is, if more than one Compound is administered, then, in some aspects, the dosages correspond to the total amount administered. In a specific aspect, oral compositions contain about 5% to about 95% of Compound 1 by weight.

The length of time that a subject in need thereof is administered Compound 1 or a pharmaceutical composition thereof in accordance with a method for preventing, treating or ameliorating a multiple myeloma in a subject in need thereof will be the time period that is determined by cancer free survival or freedom from symptoms. In certain aspects, a method for treating a multiple myeloma presented herein comprises the administration of Compound 1 or a pharmaceutical composition thereof for a period of time until the severity and/or number of one or more symptoms associated with the multiple myeloma decreases.

In some aspects, a method for preventing, treating or ameliorating a multiple myeloma in a subject in need thereof comprises the administration of Compound 1 or a pharmaceutical composition thereof for up to 48 weeks. In other aspects, a method for preventing, treating or ameliorating a multiple myeloma in a subject in need thereof comprises the administration of Compound 1 or a pharmaceutical composition thereof for up to 4 weeks, 8 weeks, 12 weeks, 16 weeks, 20 weeks, 24 weeks, 26 weeks (0.5 year), 52 weeks (1 year), 78 weeks (1.5 years), 104 weeks (2 years), or 130 weeks (2.5 years) or more.

In certain aspects, a method for preventing, treating or ameliorating a multiple myeloma in a subject in need thereof comprises the administration of Compound 1 or a pharmaceutical composition thereof for an indefinite period of time. In some aspects, a method for treating a multiple myeloma presented herein comprises the administration of Compound 1 or a pharmaceutical composition thereof for a period of time followed by a period of rest (i.e., a period wherein Compound 1 or a pharmaceutical composition thereof is not administered) before the administration of Compound 1 or a pharmaceutical composition thereof is resumed.

In specific aspects, a method for preventing, treating or ameliorating a multiple myeloma in a subject in need thereof comprises the administration of Compound 1 or a pharmaceutical composition thereof in cycles, e.g., 1 week cycles, 2 week cycles, 3 week cycles, 4 week cycles, 5 week cycles, 6 week cycles, 8 week cycles, 9 week cycles, 10 week cycles, 11 week cycles, or 12 week cycles. In such cycles, Compound 1 or a pharmaceutical composition thereof may be administered once or twice per week. In a specific aspect of a weekly cycle, Compound 1 or a pharmaceutical composition thereof may be administered twice per week. In a specific aspect of such a weekly cycle, Compound 1 or a pharmaceutical composition thereof may be administered once per day.

In specific aspects, the period of time of administration of Compound 1 or a pharmaceutical composition thereof may be dictated by one or more monitoring parameters, e.g., concentration of certain biomarkers.

In particular aspects, the period of time of administration of Compound 1 or a pharmaceutical composition thereof may be adjusted based on one or more monitoring parameters, e.g., concentration of biomarkers.

In certain aspects, in accordance with a method for preventing, treating or ameliorating a multiple myeloma in a subject in need thereof, Compound 1 or a pharmaceutical composition thereof is administered to a subject in need thereof prior to, concurrently with, or after a meal (e.g., breakfast, lunch, or dinner). In specific aspects, in accordance with the methods for treating a multiple myeloma presented herein, Compound 1 or a pharmaceutical composition thereof is administered to a subject in need thereof in the morning (e.g., between 5 am and 12 pm).

In certain aspects, in accordance with a method for preventing, treating or ameliorating a multiple myeloma in a subject in need thereof, Compound 1 or a pharmaceutical composition thereof is administered to a subject in need thereof at noon (i.e., 12 pm). In particular aspects, in accordance with the methods for treating a multiple myeloma presented herein, Compound 1 or a pharmaceutical composition thereof is administered to a subject in need thereof in the afternoon (e.g., between 12 pm and 5 pm), evening (e.g., between 5 pm and bedtime), and/or before bedtime.

In a specific aspect, a dose of Compound 1 or a pharmaceutical composition thereof is administered to a subject once per day and twice per week.

Combination Therapies Presented herein are combination therapies for the treatment of a multiple myeloma which involve the administration of Compound 1 or a pharmaceutical composition thereof in combination with one or more additional therapies to a subject in need thereof. In a specific aspect, presented herein are combination therapies for the treatment of a multiple myeloma which involve the administration of an effective amount of Compound 1 or a pharmaceutical composition thereof in combination with an effective amount of another therapy to a subject in need thereof.

As used herein, the term “in combination,” refers, in the context of the administration of Compound 1 or a pharmaceutical composition thereof, to the administration of Compound 1 or a pharmaceutical composition thereof prior to, concurrently with, or subsequent to the administration of one or more additional therapies (e.g., agents, surgery, or radiation) for use in treating a multiple myeloma. The use of the term “in combination” does not restrict the order in which one or more therapeutic agents and one or more additional therapies are administered to a subject. In specific aspects, the interval of time between the administration of Compound 1 or a pharmaceutical composition thereof and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months 5 months, 6 months, 7 months, 8 months, 9 months, 10 months, 11 months, 12 months, 1 year, 2 years, or any period of time in between. In certain aspects, Compound 1 or a pharmaceutical composition thereof and one or more additional therapies are administered less than 1 day, 1 week, 2 weeks, 3 weeks, 4 weeks, one month, 2 months, 3 months, 6 months, 1 year, 2 years, or 5 years apart.

In some aspects, the combination therapies provided herein involve administering Compound 1 or a pharmaceutical composition thereof daily, and administering one or more additional therapies once a week, once every 2 weeks, once every 3 weeks, once every 4 weeks, once every month, once every 2 months (e.g., approximately 8 weeks), once every 3 months (e.g., approximately 12 weeks), or once every 4 months (e.g., approximately 16 weeks). In certain aspects, Compound 1 or a pharmaceutical composition thereof and one or more additional therapies are cyclically administered to a subject. Cycling therapy comprises the administration of Compound 1 or a pharmaceutical composition thereof for a period of time, followed by the administration of one or more additional therapies for a period of time, and repeating this sequential administration. In certain aspects, cycling therapy may also include a period of rest where Compound 1 or a pharmaceutical composition thereof or the additional therapy is not administered for a period of time (e.g., 2 days, 3 days, 4 days, 5 days, 6 days, 7 days, 1 week, 2 weeks, 3 weeks, 4 weeks, 5 weeks, 10 weeks, 20 weeks, 1 month, 2 months, 3 months, 4 months, 5 months, 6 months, 7 months, 8 months, 9 months, 10 months, 11 months, 12 months, 2 years, or 3 years). In an aspect, the number of cycles administered is from 1 to 12 cycles, from 2 to 10 cycles, or from 2 to 8 cycles.

In some aspects, a method for preventing, treating or ameliorating a multiple myeloma in a subject in need thereof comprises administering Compound 1 or a pharmaceutical composition thereof as a single agent for a period of time prior to administering Compound 1 or a pharmaceutical composition thereof in combination with an additional therapy. In certain aspects, the methods for treating a multiple myeloma provided herein comprise administering an additional therapy alone for a period of time prior to administering Compound 1 or a pharmaceutical composition thereof in combination with the additional therapy.

In some aspects, the administration of Compound 1 or a pharmaceutical composition thereof and one or more additional therapies in accordance with the methods presented herein have an additive effect relative the administration of Compound 1 or a pharmaceutical composition thereof or said one or more additional therapies alone. In some aspects, the administration of Compound 1 or a pharmaceutical composition thereof and one or more additional therapies in accordance with the methods presented herein have a synergistic effect relative to the administration of Compound 1 or a pharmaceutical composition thereof or said one or more additional therapies alone.

As used herein, the term “synergistic,” refers to the effect of the administration of Compound 1 or a pharmaceutical composition thereof in combination with one or more additional therapies (e.g., agents), which combination is more effective than the additive effects of any two or more single therapies (e.g., agents).

In a specific aspect, a synergistic effect of a combination therapy permits the use of lower dosages (i.e., sub-optimal doses) of Compound 1 or a pharmaceutical composition thereof or an additional therapy and/or less frequent administration of Compound 1 or a pharmaceutical composition thereof or an additional therapy to a subject.

In certain aspects, the ability to utilize lower dosages of Compound 1 or a pharmaceutical composition thereof or of an additional therapy and/or to administer Compound 1 or a pharmaceutical composition thereof or said additional therapy less frequently reduces the toxicity associated with the administration of Compound 1 or a pharmaceutical composition thereof or of said additional therapy, respectively, to a subject without reducing the efficacy of Compound 1 or a pharmaceutical composition thereof or of said additional therapy, respectively, in the treatment of a multiple myeloma.

In some aspects, a synergistic effect results in improved efficacy of Compound 1 or a pharmaceutical composition thereof and each of said additional therapies in treating a multiple myeloma. In some aspects, a synergistic effect of a combination of Compound 1 or a pharmaceutical composition thereof and one or more additional therapies avoids or reduces adverse or unwanted side effects associated with the use of any single therapy.

The combination of Compound 1 or a pharmaceutical composition thereof and one or more additional therapies can be administered to a subject in the same pharmaceutical composition. Alternatively, Compound 1 or a pharmaceutical composition thereof and one or more additional therapies can be administered concurrently to a subject in separate pharmaceutical compositions. Compound 1 or a pharmaceutical composition thereof and one or more additional therapies can be administered sequentially to a subject in separate pharmaceutical compositions. Compound 1 or a pharmaceutical composition thereof and one or more additional therapies may also be administered to a subject by the same or different routes of administration.

The combination therapies provided herein involve administering to a subject to in need thereof Compound 1 or a pharmaceutical composition thereof in combination with conventional, or known, therapies for treating a multiple myeloma. Other therapies for a multiple myeloma or a condition associated therewith are aimed at controlling or relieving one or more symptoms. Accordingly, in some aspects, the combination therapies provided herein involve administering to a subject to in need thereof a pain reliever, or other therapies aimed at alleviating or controlling one or more symptoms associated with a multiple myeloma or a condition associated therewith.

Specific examples of anti-cancer agents that may be used in combination with Compound 1 or a pharmaceutical composition thereof for treating a multiple myeloma include: a hormonal agent (e.g., aromatase inhibitor, selective estrogen receptor modulator (SERM), and estrogen receptor antagonist), chemotherapeutic agent (e.g., microtubule dissembly blocker, antimetabolite, topisomerase inhibitor, and DNA crosslinker or damaging agent), anti-angiogenic agent (e.g., VEGF antagonist, receptor antagonist, integrin antagonist, vascular targeting agent (VTA)/vascular disrupting agent (VDA)), radiation therapy, and conventional surgery.

Non-limiting examples of hormonal agents that may be used in combination with Compound 1 or a pharmaceutical composition thereof for treating a multiple myeloma include aromatase inhibitors, SERMs, and estrogen receptor antagonists. Hormonal agents that are aromatase inhibitors may be steroidal or nonsteroidal. Non-limiting examples of nonsteroidal hormonal agents include letrozole, anastrozole, aminoglutethimide, fadrozole, and vorozole. Non-limiting examples of steroidal hormonal agents include aromasin (exemestane), formestane, and testolactone. Non-limiting examples of hormonal agents that are SERMs include tamoxifen (branded/marketed as Nolvadex®), afimoxifene, arzoxifene, bazedoxifene, clomifene, femarelle, lasofoxifene, ormeloxifene, raloxifene, and toremifene. Non-limiting examples of hormonal agents that are estrogen receptor antagonists include fulvestrant. Other hormonal agents include but are not limited to abiraterone and lonaprisan.

Non-limiting examples of chemotherapeutic agents that may be used in combination with Compound 1 or a pharmaceutical composition thereof for treating cancer include microtubule disassembly blocker, antimetabolite, topoisomerase inhibitor, and DNA crosslinker or damaging agent.

Chemotherapeutic agents that are microtubule disassembly blockers include, but are not limited to, taxenes (e.g., paclitaxel (branded/marketed as TAXOL®), docetaxel, nabPaclitaxel (branded/marketed as ABRAXANE®), larotaxel, ortataxel, and tesetaxel); epothilones (e.g., ixabepilone); and vincalkaloids (e.g., vinorelbine, vinblastine, vindesine, and vincristine (branded/marketed as ONCOVIN®)).

Chemotherapeutic agents that are antimetabolites include, but are not limited to, folate antimetabolites (e.g., methotrexate, aminopterin, pemetrexed, raltitrexed); purine antimetabolites (e.g., cladribine, clofarabine, fludarabine, mercaptopurine, pentostatin, thioguanine); pyrimidine antimetabolites (e.g., 5-fluorouracil, capcitabine, gemcitabine (GEMZAR®), cytarabine, decitabine, floxuridine, tegafur); and deoxyribonucleotide antimetabolites (e.g., hydroxyurea).

Chemotherapeutic agents that are topoisomerase inhibitors include, but are not limited to, class I (camptotheca) topoisomerase inhibitors (e.g., topotecan (branded/marketed as HYCAMTIN®) irinotecan, rubitecan, and belotecan); class II (podophyllum) topoisomerase inhibitors (e.g., etoposide or VP-16, and teniposide); anthracyclines (e.g., doxorubicin, epirubicin, Doxil, aclarubicin, amrubicin, daunorubicin, idarubicin, pirarubicin, valrubicin, and zorubicin); and anthracenediones (e.g., mitoxantrone, and pixantrone).

Chemotherapeutic agents that are DNA crosslinkers (or DNA damaging agents) include, but are not limited to, alkylating agents (e.g., cyclophosphamide, mechlorethamine, ifosfamide (branded/marketed as IFEX®), trofosfamide, chlorambucil, melphalan, prednimustine, bendamustine, uramustine, estramustine, carmustine (branded/marketed as BiCNU®), lomustine, semustine, fotemustine, nimustine, ranimustine, streptozocin, busulfan, mannosulfan, treosulfan, carboquone, N,N′N′-triethylenethiophosphoramide, triaziquone, triethylenemelamine); alkylating-like agents (e.g., carboplatin (branded/marketed as PARAPLATIN®), cisplatin, oxaliplatin, nedaplatin, triplatin tetranitrate, satraplatin, picoplatin); nonclassical DNA crosslinkers (e.g., procarbazine, dacarbazine, temozolomide (branded/marketed as TEMODAR®), altretamine, mitobronitol); and intercalating agents (e.g., actinomycin, bleomycin, mitomycin, and plicamycin).

Non-limiting examples of anti-angiogenic agents that may be used in combination with Compound 1 or a pharmaceutical composition thereof for treating a multiple myeloma include VEGF antagonists, receptor antagonists, integrin antagonists (e.g., vitaxin, cilengitide, and S247), and VTAs/VDAs (e.g., fosbretabulin). VEGF antagonists include, but are not to, anti-VEGF antibodies (e.g., bevacizumab (branded/marketed as AVASTIN®) and ranibizumab (branded/marketed as LUCENTIS®)), VEGF traps (e.g., aflibercept), VEGF antisense or siRNA or miRNA, and aptamers (e.g., pegaptanib (branded/marketed as MACUGEN®)). Anti-angiogenic agents that are receptor antagonists include, but are not limited to, antibodies (e.g., ramucirumab) and kinase inhibitors (e.g., sunitinib, sorafenib, cediranib, panzopanib, vandetanib, axitinib, and AG-013958) such as tyrosine kinase inhibitors. Other non-limiting examples of anti-angiogenic agents include ATN-224, anecortave acetate (branded/marketed as RETAANE®), microtubule depolymerization inhibitor such as combretastatin A4 prodrug, and protein or protein fragment such as collagen 18 (endostatin).

Non-limiting examples of other therapies that may be administered to a subject in combination with Compound 1 or a pharmaceutical composition thereof for treating a multiple myeloma include:

(1) a statin such as lovostatin (e.g., branded/marketed as MEVACOR®);

(2) an mTOR inhibitor such as sirolimus which is also known as Rapamycin (e.g., branded/marketed as RAPAMUNE®), temsirolimus (e.g., branded/marketed as TORISEL®), evorolimus (e.g., branded/marketed as AFINITOR®), and deforolimus;

(3) a farnesyltransferase inhibitor agent such as tipifarnib (e.g., branded/marketed as ZARNESTRA®);

(4) an antifibrotic agent such as pirfenidone;

(5) a pegylated interferon such as PEG-interferon alfa-2b;

(6) a CNS stimulant such as methylphenidate (branded/marketed as RITALIN®);

(7) a HER-2 antagonist such as anti-HER-2 antibody (e.g., trastuzumab) and kinase inhibitor (e.g., lapatinib);

(8) an IGF-1 antagonist such as an anti-IGF-1 antibody (e.g., AVE1642 and IMC-A11) or an IGF-1 kinase inhibitor;

(9) EGFR/HER-1 antagonist such as an anti-EGFR antibody (e.g., cetuximab, panitumamab) or EGFR kinase inhibitor (e.g., erlotinib (e.g., branded/marketed as TARCEVA®), gefitinib);

(10) SRC antagonist such as bosutinib;

(11) cyclin dependent kinase (CDK) inhibitor such as seliciclib;

(12) Janus kinase 2 inhibitor such as lestaurtinib;

(13) proteasome inhibitor such as bortezomib;

(14) phosphodiesterase inhibitor such as anagrelide;

(15) inosine monophosphate dehydrogenase inhibitor such as tiazofurine;

(16) lipoxygenase inhibitor such as masoprocol;

(17) endothelin antagonist;

(18) retinoid receptor antagonist such as tretinoin or alitretinoin;

(19) immune modulator such as lenalidomide, pomalidomide, or thalidomide (e.g., branded/marketed as THALIDOMID®);

(20) kinase (eg, tyrosine kinase) inhibitor such as imatinib (e.g., branded/marketed as GLEEVEC®), dasatinib, erlotinib, nilotinib, gefitinib, sorafenib, sunitinib (e.g., branded/marketed as SUTENT®), lapatinib, AEE788, or TG100801;

(21) non-steroidal anti-inflammatory agent such as celecoxib (branded/marketed as CELEBREX®);

(22) human granulocyte colony-stimulating factor (G-CSF) such as filgrastim (branded/marketed as NEUPOGEN®);

(23) folinic acid or leucovorin calcium;

(24) integrin antagonist such as an integrin α5β1-antagonist (e.g., JSM6427);

(25) nuclear factor kappa beta (NF-κβ) antagonist such as OT-551, which is also an anti-oxidant;

(26) hedgehog inhibitor such as CUR61414, cyclopamine, GDC-0449, or anti-hedgehog antibody;

(27) histone deacetylase (HDAC) inhibitor such as SAHA (also known as vorinostat (branded/marketed as ZOLINZA®)), PCI-24781, SB939, CHR-3996, CRA-024781, ITF2357, JNJ-26481585, or PCI-24781;

(28) retinoid such as isotretinoin (e.g., branded/marketed as ACCUTANE®);

(29) hepatocyte growth factor/scatter factor (HGF/SF) antagonist such as HGF/SF monoclonal antibody (e.g., AMG 102);

(30) synthetic chemical such as antineoplaston;

(31) anti-diabetic such as rosiglitazone maleate (e.g., branded/marketed as AVANDIA®);

(32) antimalarial and amebicidal drug such as chloroquine (e.g., branded/marketed as ARALEN®);

(33) synthetic bradykinin such as RMP-7;

(34) platelet-derived growth factor receptor inhibitor such as SU-101;

(35) receptor tyrosine kinase inhibitors of Flk-1/KDR/VEGFR2, FGFR1 and PDGFR beta such as SU5416 and SU6668;

(36) anti-inflammatory agent such as sulfasalazine (e.g., branded/marketed as AZULFIDINE®); and

(37) TGF-beta antisense therapy.

Non-limiting examples of other therapies that may be administered to a subject in combination with Compound 1 or a pharmaceutical composition thereof for treating a multiple myeloma include: a synthetic nonapeptide analog of naturally occurring gonadotropin releasing hormone such as leuprolide acetate (branded/marketed as LUPRON®); a nonsteroidal, anti-androgen such as flutamide (branded/marketed as EULEXIN®) or nilutamide (branded/marketed as NILANDRON®); a non-steroidal androgen receptor inhibitor such as bicalutamide (branded/marketed as CASODEX®); steroid hormone such as progesterone; anti-fungal agent such as Ketoconazole (branded/marketed as NIZORAL®); glucocorticoid such as prednisone; estramustine phosphate sodium (branded/marketed as EMCYT®); and bisphosphonate such as pamidronate, alendronate, and risedronate.

Additional specific examples of therapies that may be used in combination with Compound 1 or a pharmaceutical composition thereof for treating a multiple myeloma include, but are not limited to, agents associated with cancer immunotherapy (e.g., cytokines, interleukins, and cancer vaccines).

Specific examples of agents alleviating side-effects associated with a multiple myeloma that can be used as therapies in combination with Compound 1 or a pharmaceutical composition thereof, include, but are not limited to: antiemetics, e.g., Ondansetron hydrochloride (branded/marketed as ZOFRAN®), Granisetron hydrochloride (branded/marketed as KYTRIL®), Lorazepam (branded/marketed as ATIVAN®) and Dexamethasone (branded/marketed as DECADRON®).

In certain aspects, combination therapies provided herein for treating a multiple myeloma comprise administering Compound 1 or a pharmaceutical composition thereof in combination with one or more agents used to treat and/or manage a side effect, such as, bleeding (usually transient, low-grade epistaxis), arterial and venous thrombosis, hypertension, delayed wound healing, asymptomatic proteinuria, nasal septal perforation, reversible posterior leukoencephalopathy syndrome in association with hypertension, light-headedness, ataxia, headache, hoarseness, nausea, vomiting, diarrhea, rash, subungual hemorrhage, myelodysplastic syndromes, myelosuppression, fatigue, hypothyroidism, QT interval prolongation, or heart failure.

In certain aspects, Compound 1 or a pharmaceutical composition thereof is not used in combination with a drug that is primarily metabolized by CYP2D6 (such as an antidepressant (e.g, a atricyclic antidepressant, a selective serotonin reuptake inhibitor, and the like), an antipsychotic, a beta-adrenergic receptor blocker, or certain types of anti-arrhythmics) to treat a multiple myeloma.

### Kits

Provided herein is a pharmaceutical pack or kit comprising one or more containers filled with Compound 1 or a pharmaceutical composition thereof. Additionally, one or more other therapies useful for the treatment of a multiple myeloma, or other relevant agents can also be included in the pharmaceutical pack or kit. Also provided herein is a pharmaceutical pack or kit comprising one or more containers filled with one or more of the ingredients of the pharmaceutical compositions described herein. Optionally associated with such kits can be a notice in the form prescribed by a governmental agency regulating the manufacture, use or sale of pharmaceuticals or biological products, which notice reflects approval by the agency of manufacture, use or sale for human administration.

### EXAMPLES

### Example 1

Compound 1 was tested for usefulness in affecting MM proliferation using a comprehensive set of in vitro and in vivo models.

### Methods

The effect of Compound 1 was tested in human MM cell lines (HMCLs) using cytotoxicity, colony formation, co-culture, qPCR, Western Blot, flow cytometry, ELISA, and lentiviral transduction experiments. The effect of Compound 1 was assessed in vivo in the 5TGM1 murine model of MM.

### Results

The effect of Compound 1 downregulated BMI-1 protein expression within 24 hrs of treatment and demonstrated potent activity in parental and PI-resistant HMCLs (n=16), having a median IC₅₀ 57.2 nM and corresponding fold-decrease (R>0.8, P<0.01). Similar potency was observed in co-culture and colony formation assays.

While Compound 1 did not rescue the MM cells from BMI-1 overexpression, time course experiments demonstrated potent mitotic arrest 6-24 h post treatment associated with elevated expression of Cyclin B1, AURKA and BIRC5 as well as downregulation of MCL1. Prolonged mitosis was followed by the induction of apoptosis verified by the presence of Annexin V positive cells, cleaved caspases 8 and 9, cleaved PARP, loss of MCL1 protein and depolarization of the mitochondrial membrane potential.

Since Compound 1 is known to cause mitotic arrest, central MM signalling cascades were found to lead to a significant reduction of MYC and AKT activity (in contrast to unaffected ERK and GSK3b). Downregulation of MYC and FOXM1 protein expression demonstrated that Compound 1 can affect the proliferative activity of key MM genes.

Drug combination studies showed synergism with established drugs (IMiDs, Dex, PIs, MEL) and BH3 mimetics (targeting BCL2, BCLxL, MCL1) in individual cell lines. Consistent synergism was observed with epigenetic modulators (targeting EZH2, CBP/EP300, BRD4, HDACs) suggesting that impaired PRC-1 activity due to loss of BMI-1 might prone MM cells to treatment with a combination therapy of Compound 1 and an epigenetic drug.

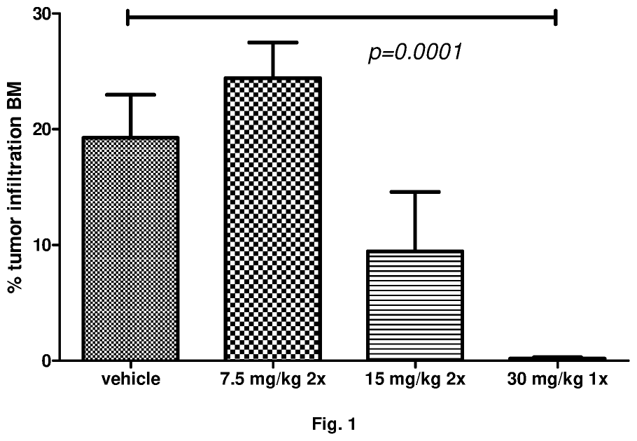

The in vivo activity of Compound 1 in the 5TGM1 model further demonstrated a reproducible dose dependent reduction of BM infiltration (as shown in FIG. 1), with complete eradication of MM cells by treatment with Compound 1 at 30 mg/kg/biweekly.

### CONCLUSION

Compound 1 has demonstrated promising pre-clinical activity for the treatment of MM, as either a primary or secondary (i.e., combination) therapy. Moreover, the data suggests the use reduced BMI-1 protein levels as a predictive biomarker. As a potent anti-mitotic agent, Compound 1 has demonstrated an ability to target key MM genes (e.g., MYC) and synergistic activity with epigenetic compounds. These results strongly demonstrate the potential therapeutic utility of Compound 1 for the treatment of multiple myeloma.

### REFERENCES

1. Targeting of BMI-1 with PTC-209 shows potent anti-myeloma activity and impairs the tumour microenvironment; Bolomsky A, Schlangen K, Schreiner W, Zojer N, Ludwig H; J. Hematol. Oncol. 2016, Mar. 2; 9:17

Without regard to whether a document cited herein was specifically and individually indicated as being incorporated by reference, all documents referred to herein are incorporated by reference into the present application for any and all purposes to the same extent as if each individual reference was fully set forth herein.

Having now fully described the subject matter of the claims, it will be understood by those having ordinary skill in the art that the same can be performed within a wide range of equivalents without affecting the scope of the subject matter or aspects described herein. It is intended that the appended claims be interpreted to include all such equivalents.

What is claimed is:

1. A method for treating a multiple myeloma in a subject in need thereof comprising, administering to the subject an effective amount of 5-fluoro-2-(6-fluoro-2-methyl-1H-benzo[d]imidazol-1-yl)-N⁴-[4-(trifluoromethyl)phenyl]pyrimidine-4,6-diamine, having the structure of Formula (1): 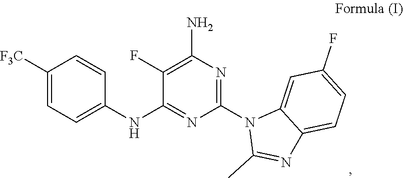 [SMILES: Cc1nc2ccc(F)cc2n1-c1nc(N)c(F)c(Nc2ccc(C(F)(F)F)cc2)n1]
or a pharmaceutically acceptable salt or pharmaceutical composition thereof.

2. The method of claim 1, further comprising, administering to the subject an effective amount of 5-fluoro-2-(6-fluoro-2-methyl-1H-benzo[d]imidazol-1-yl)-N⁴-[4-(trifluoromethyl)phenyl]pyrimidine-4,6-diamine or a pharmaceutically acceptable salt or pharmaceutical composition thereof in combination with an effective amount of one or more chemotherapeutic agents.
